(19) United States
(12) Patent Application Publication
(10) Pub. No.: US 2019/0271503 A1
(43) Pub. Date: Sep. 5, 2019

(54) PROCESS FOR RECOVERING PROPANE AND AN ADJUSTABLE AMOUNT OF ETHANE FROM NATURAL GAS

(71) Applicant: L'Air Liquide, Société Anonyme pour I'Etude et I'Exploitation des Procédés Georges Claude, Paris (FR)

(72) Inventors: Paul TERRIEN, Newton, MA (US); Henri Paradowski, Pluvigner (FR); Jonathan Li, Champigney-Sur-Marne (FR)

(21) Appl. No.: 16/156,127

(22) Filed: Oct. 10, 2018

(30) Foreign Application Priority Data
Oct. 10, 2017 (FR) 1701049

(51) Int. Cl. F25J 3/02; C07C 7/04; C10L 3/10

(52) U.S. Cl. CPC F25J 3/0209; C10L 2290/543; F25J 3/0233; C07C 7/04; C10L 3/101; F25J 2270/60; F25J 2200/02; F25J 2200/04; F25J 2200/70; F25J 2200/76; F25J 2200/78; F25J 2205/04; F25J 2210/06; F25J 2200/00; F25J 2230/60; F25J 2240/02; F25J 2270/12; F25J 3/0238

(57) ABSTRACT

A process for simultaneously producing treated natural gas and a propane-rich stream from a feed gas comprising methane, ethane and hydrocarbons having more than three carbon atoms.

### CROSS REFERENCE TO RELATED APPLICATIONS

This application claims the benefit of priority under 35 U.S.C. § 119 (a) and (b) to French Patent Application No. 1701049 filed Oct. 10, 2017, the entire contents of which are incorporated herein by reference.

### BACKGROUND

The present invention relates to a process for the simultaneous production of treated natural gas from a starting natural gas comprising methane, ethane and C3+ hydrocarbons of a fraction rich in heavy hydrocarbons. This fraction rich in heavy hydrocarbons contains the C3+ hydrocarbons (that is to say the hydrocarbons having at least three carbon atoms) and, in at least some production conditions, also contains ethane (at least 5 mol %).

Optimized processes for simultaneously extracting virtually all the C3+ hydrocarbons in the starting natural gas, and a high proportion of ethane of the starting gas, are known. Thus, when the degree of ethane extraction is at least 70%, the degree of propane extraction is close to 99%. Such processes are not entirely satisfactory.

This is because the demand for ethane on the market fluctuates a great deal, whereas the demand for C3+ hydrocarbon fractions remains relatively constant and well exploited. It is then sometimes necessary to reduce the production of ethane in the process, by reducing the degree of extraction of this compound. In this case, the degree of extraction of the C3+ hydrocarbons also decreases, which reduces the profitability of the facility.

Document U.S. Pat. No. 7,484,385 describes a solution for separating C2+ hydrocarbons from natural gas. It is possible to obtain high ethane yields according to certain production conditions. On the other hand, this solution does not allow flexibility making it possible to have two efficient modes:

a mode designed to obtain a high degree of ethane extraction, and

a mode designed to obtain a low degree of ethane extraction.

Document U.S. Pat. No. 7,458,232 describes a flexible solution for recovering either the C2+ products or the C3+ products. On the other hand, the process described enables only a very moderate ethane recovery.

The inventors of the present invention have therefore developed a solution making it possible to solve the problems raised above.

### SUMMARY

An objective of the present invention is to provide a process which makes it possible, by simple and inexpensive means, to extract substantially all of the C3+ hydrocarbons from a stream of starting natural gas, regardless of the amount of ethane produced by the process and while ensuring an ethane recovery that is higher than in the implementation of the processes described in the prior art in an “ethane recovery” mode, combined with a totally flexible operation which allows very little ethane recovery while at the same time keeping a high propane recovery in “ethane discard” mode.

A subject of the present invention is a process for simultaneously producing treated natural gas and a propane-rich stream from a feed gas comprising methane, ethane and hydrocarbons having more than three carbon atoms, said process comprising the following steps:

Step a): the feed gas is cooled and partially condensed;

Step b): the cooled gas resulting from step a) is separated into a first liquid stream and a first gas stream by means of a first phase separator vessel at a temperature T1 and a pressure P1;

Step c): at least one portion of the first gas stream resulting from step b) is expanded by means of an expansion means;

Step d): the expanded gas resulting from step c) is introduced into a first distillation column at a first intermediate level N′;

Step e): a bottom liquid fraction is recovered from said first distillation column and is introduced into a second distillation column at a feed level M1;

characterized in that it comprises one or other of the following steps depending on the desired degree of ethane in the streams produced:

Step f): in order to obtain degrees of ethane extraction greater than a first predetermined threshold, at least one portion of said first gas stream resulting from step b) is partially condensed and is introduced into a second phase separator vessel at a pressure P2 and a temperature T2 in order to produce a second gas stream and a second liquid stream, at least one portion of said second gas stream is condensed and introduced into said first distillation column at a level S1 above the level N′;

Step g): in order to obtain degrees of ethane extraction below a second predetermined threshold, a gas fraction is recovered at the top of said second distillation column, and is then condensed before introducing it into said first distillation column at the level S1 above the level N′.

According to other embodiments, subjects of the invention are also:

A process as defined above, characterized in that P2 is lower than P1 and T2 is lower than T1.

A process as defined above, characterized in that step f) also comprises step f1): at least one portion of said second gas stream resulting from the second phase separator vessel is partially condensed and is introduced into a third phase separator vessel at a pressure P3 and a temperature T3 in order to produce a third gas stream and a third liquid stream, at least one portion of said third gas stream is condensed and introduced into said first distillation column at the level S1 above the level N′.

A process as defined above, characterized in that P1<P2<P3 and T1<T2<T3.

A process as defined above, characterized in that said first predetermined threshold is greater than or equal to 80%.

A process as defined above, characterized in that said second predetermined threshold is less than or equal to 20%.

A process as defined above, characterized in that said propane-rich stream comprises at least 99.5% of the propane initially contained in the feed stream.

A process as defined above, characterized in that said ethane-rich stream comprises at least 95% of the ethane initially contained in the feed stream.

A process as defined above, characterized in that a portion of the gas fraction from the top of the second distillation column is condensed in a heat exchanger by circulation of a portion of the gas from the top of the first distillation column.

A process as defined above, in which:

during step a), the feed gas is at least partially condensed in a first heat exchanger; a liquid stream is extracted from the first distillation column at an intermediate level S2 lower than the level N″ and is partially vaporized in a second heat exchanger distinct from said first heat exchanger;

said liquid fraction recovered during step e) is pumped into and then at least partially vaporized in said second heat exchanger; and

a fraction of the feed gas is cooled in said second heat exchanger.

A subject of the present invention is also:

A facility, for carrying out the process as defined above, for simultaneously producing treated natural gas and a propane-rich stream from a feed gas comprising methane, ethane and hydrocarbons having more than three carbon atoms, said process comprising:

a first heat exchanger for cooling to condense a feed gas;

a first phase separator vessel for separating the gas cooled in the first condensation means into a first liquid stream and a first gas stream;

a first distillation column into which at least one portion of the first gas stream is introduced at a first intermediate level N′;

a second distillation column into which a liquid fraction originating from the bottom of said first distillation column is introduced at at least one feed level M1, M2;

characterized in that it comprises means for producing a stream, having a degree of ethane recovery above a predetermined threshold, originating from a second phase separator vessel, located downstream of the first phase separator vessel, producing a second gas stream and a second liquid stream, at least one portion of said second gas stream being condensed and introduced into said first distillation column at a level S1 above the level N′; and

characterized in that it comprises means for producing a stream, having a degree of ethane recovery below a second predetermined threshold, originating from a gas fraction at the top of said second distillation column, then introduced into said first distillation column at the level S1 above the level N′.

A facility as defined above, characterized in that it comprises a third phase separator vessel, located downstream of the second phase separator vessel, producing a third gas stream and a third liquid stream, at least one portion of said third gas stream being condensed and introduced into said first distillation column at the level S1 above the level N′.

A facility as defined above, characterized in that it comprises a second heat exchanger capable of and designed for:

partially vaporizing a liquid stream extracted from the first distillation column at an intermediate level S2 below the level N″ and also a liquid fraction recovered at the bottom of said first distillation column; and

cooling and at least partially condensing a fraction of the feed gas.

The process which is the subject of the present invention uses two distillation columns for the optimized recovery of the C3+ hydrocarbon products or of the C2+ hydrocarbon products. The thermal integration (optimized heat exchangers) makes it possible to maximize the performances of the process. In addition, the process which is the subject of the present invention implements successive partial and expanded condensations in order to optimize the refluxes provided in the first distillation column during step f). It is important to optimize the operating pressure and temperature of each of the phase separator vessels in order to optimize the performances.

The natural gas stream is usually composed essentially of methane. Preferably, the feed stream comprises at least 80 mol % of methane. Depending on the source, natural gas contains amounts of hydrocarbons that are heavier than methane, such as for example ethane, propane, butane and pentane, and also certain aromatic hydrocarbons. The natural gas stream also contains non-hydrocarbon products, such as H₂O, N₂, CO₂ or H₂S and other sulfur-bearing compounds, mercury and others.

The expression “natural gas” as used in the present application relates to any composition containing hydrocarbons including at least methane. This comprises a “crude” composition (composition which is prior to any treatment or washing), and also any composition having been partially, substantially or completely treated for the reduction and/or elimination of one or more compounds, including, but without being limited thereto, sulfur, carbon dioxide, water, mercury and certain heavy and aromatic hydrocarbons.

The heat exchanger may be any heat exchanger, any unit or other arrangement suitable for allowing a certain number of streams to pass through, and thus for allowing direct or indirect heat exchange between one or more lines of refrigerant fluid, and one or more feed streams.

The term “degree of ethane extraction” denotes the ratio of the partial molar flow of ethane contained in the liquid fraction at the bottom of said first distillation column recovered in step e) to the partial molar flow of ethane in the feed gas.

### BRIEF DESCRIPTION OF THE DRAWINGS

For a further understanding of the nature and objects for the present invention, reference should be made to the following detailed description, taken in conjunction with the accompanying drawings, in which like elements are given the same or analogous reference numbers and wherein:

FIG. 1 illustrates an embodiment of the invention.

FIG. 2 illustrates an embodiment of the invention.

### DESCRIPTION OF PREFERRED EMBODIMENTS

One and the same reference denotes a liquid stream and the pipe that conveys it, the pressures taken into consideration are absolute pressures and the percentages taken into consideration are molar percentages.

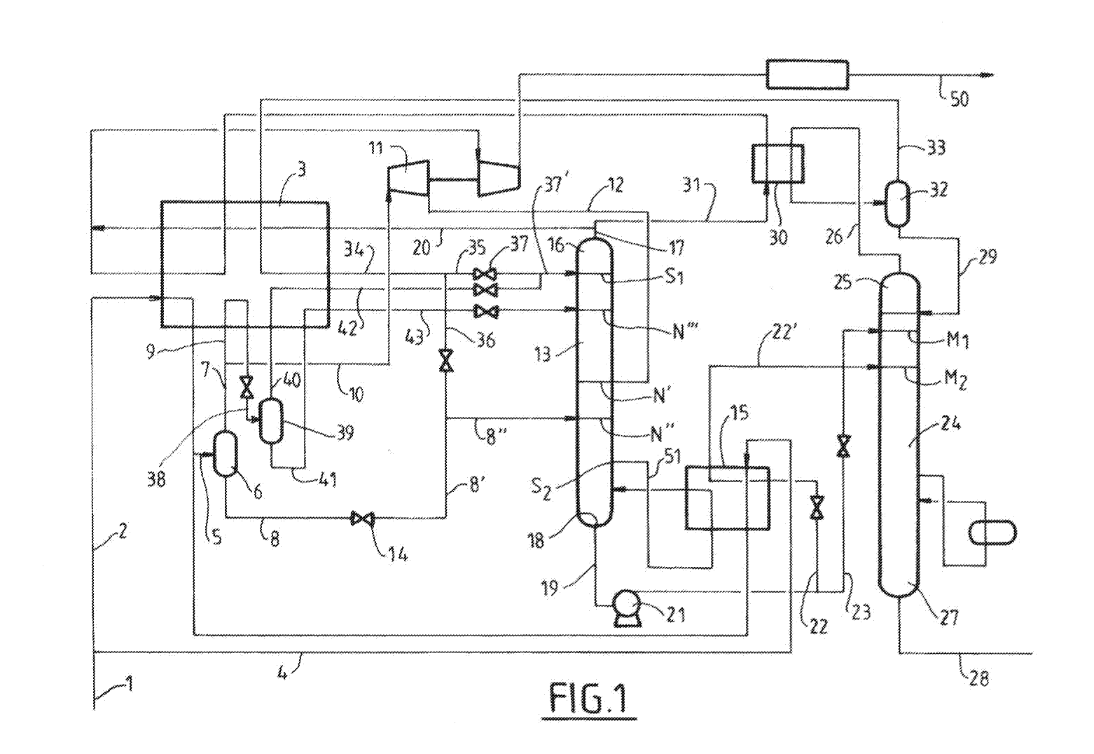

In FIG. 1, a feed stream of natural gas 1 at a pressure P0, which is generally high (greater than 20 bar a, preferentially greater than 30 bar a), and a temperature T0 of about ambient temperature is separated into two streams: a main stream 2 which is cooled in a heat exchanger 3 and a secondary stream 4. These two streams are combined to form a stream of natural gas 5 precooled at a temperature T1, which feeds a first phase separator vessel 6 producing a first gas stream 7 and a first liquid stream 8. The stream 7 is separated into two streams 9 and 10. The stream 10 feeds a “turboexpander” 11 in which it is expanded to form a stream 12 which feeds a first distillation column (or fractionation column) 13 at an intermediate introduction level N′. The liquid stream 8 is expanded 8′ in an expansion means 14 and a portion 8″ feeds the column 13 at an intermediate introduction level N″ located at a stage below the stage N′. The term intermediate level is intended to mean a position comprising distillation means above and below this level. The column 13 has a reboiler/exchanger 15 and produces, at the top 16, the treated natural gas 17 and, at the bottom 18, a mixture of liquefied gases 19. The secondary stream 4 is cooled in the reboiler 15 of the first fractionation column 13 before being mixed with the cooled stream 2. The larger portion 20 of the treated gas 17 is reheated mainly in the heat exchanger 3 up to a temperature T4 below T0. The mixture 19 is pumped by a pump 21 and then separated into two streams 22 and 23. The stream 23 is sent to a second distillation column 24 at an introduction level M1. The stream 22, after reheating, is sent 22′ to the column 24 at an introduction level M2. The secondary stream of natural gas 4 is cooled in the exchanger 15 in a direction countercurrent to the stream 22 and provides the abovementioned reheating of the stream 22′.

The column 24 produces, at the top 25, a gas mixture 26 and, at the bottom 27, a mixture of liquefied gases 28. The condensation of the reflux 29 of the column 24 is provided by a heat exchanger 30 with at least one portion 31 of the treated gas 17.

The condensation of the top stream 26 of the column 24 can be carried out in a heat exchanger 30 by circulation of a portion 31 of the stream of treated gas 17 from the top of the first column 13, which makes it possible to integrate the reflux drum 32 and the exchanger 30 above the top of the column 24 and to avoid using a reflux pump.

The drum 32 makes it possible to produce a gas stream 33 and a liquid stream 29.

The gas mixture 33 is cooled and totally condensed in the heat exchanger 3 by heat exchange with the stream 20 of treated gas 17 so as to form the mixture 34. This mixture 34 is separated into two streams 35 and 36. The stream 36 is sent to the column 13 at the introduction level N″ after mixing with the stream 8′ so as to form the stream 8″. In the interests of simplicity, this introduction is at the same introduction level N″, but it is also possible to carry out an introduction at a level close to said introduction level but distinct therefrom.

It should be noted that the stream 2 is cooled in the exchanger 3 in countercurrent mode with the treated gas 17.

The advantage of the present invention is to make it possible for the user to choose between the following two options:

Either the user decides to carry out the process in “ethane discard” mode, that is to say said user wants the stream 28 to contain a degree of ethane below a predetermined threshold;

Or the user carries out the process in “ethane recovery” mode, that is to say said user wants the stream 28 to comprise a degree of ethane above a certain predetermined threshold.

### In Ethane Discard Mode

The stream 9 has a zero or very low flow rate. This means that all or almost all the stream 7 is injected into the turboexpander 11.

The stream 34 is entirely (or at least more than half of it is) introduced at an introduction level S1 at the top of the column after expansion. The level S1 is a level above N′″, which is itself above N′.

### In Ethane Recovery Mode

The stream 9 is condensed in the exchanger 3 in countercurrent mode with the treated gas 17 and expanded so as to form, after expansion, a two-phase stream 38. This stream 38 is separated at a pressure P2 and a temperature T2, in a second separator vessel 39, into two streams: a second gas stream 40 and a second liquid stream 41. P2 is higher than P1 and T2 is lower than T1. The stream 40 is condensed in the exchanger 3 in countercurrent mode with the treated gas 17 so as to form the stream 42 which, after expansion, feeds the column 13 at the introduction level S1. The liquid 41 is subcooled in the exchanger 3 in countercurrent mode with the treated gas 17 so as to form the liquid 43 which, after expansion, feeds the column 13 at the introduction level N′″.

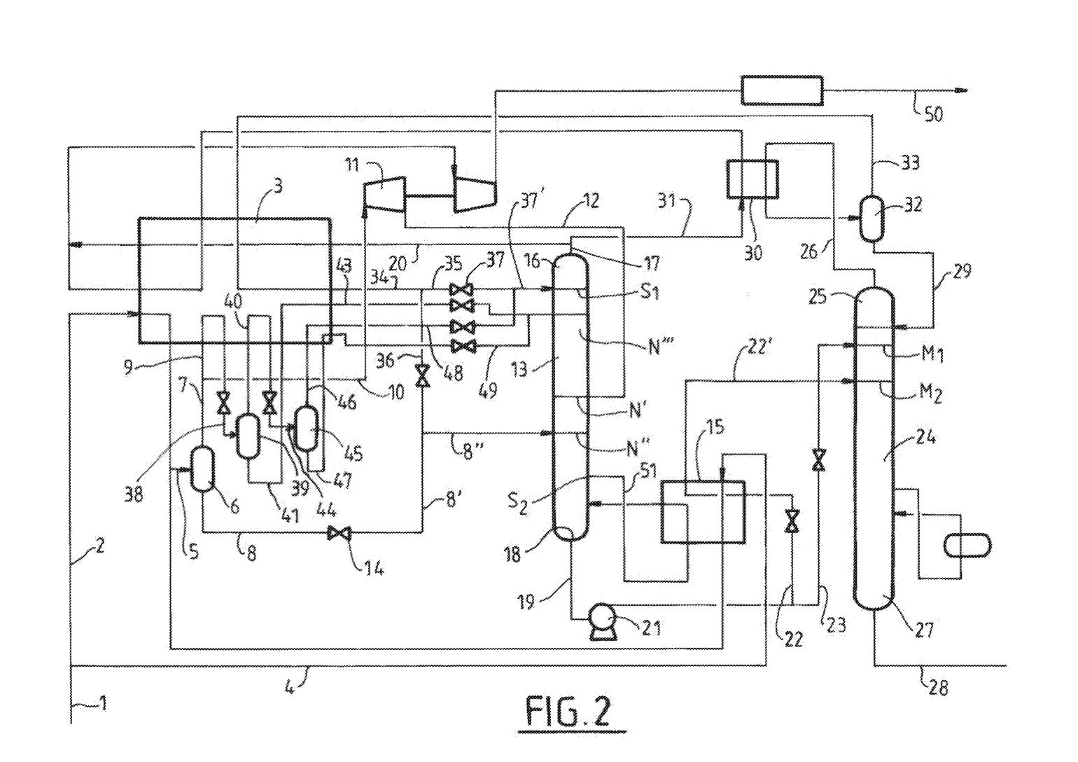

A second embodiment of the process which is the subject of the present invention is represented in FIG. 2.

The references are the same as those for FIG. 1.

The process illustrated in this FIG. 2 is similar to that which is illustrated in FIG. 1, the distinctions being the following:

### In Ethane Recovery Mode

The stream 40 is condensed in the exchanger 3 in countercurrent mode with the treated gas 17 so as to form, after expansion, a new two-phase stream 44. This stream 44 is introduced into a third phase separator vessel 45 in order to be separated, at a pressure P3 and a temperature T3, into a third gas stream 46 and a third liquid stream 47. P3 is lower than P2 and T3 is lower than T2. The stream 46 is condensed in the exchanger 3 in countercurrent mode with the treated gas 17 so as to form the stream 48 which, after expansion, feeds the column 13 at the introduction level S1 in order to form a reflux from the top of the column 13. The liquid 47 is subcooled in the exchanger 3 in countercurrent mode with the treated gas 17 so as to form the liquid 49 which, after expansion, feeds the column 13 at the introduction level N′″.

### In Ethane Discard Mode

The operating principle does not change compared with the process described according to FIG. 1 in this same “ethane discard” mode, the stream 35 being represented as a dashed line in FIG. 2.

On both figures, the final treated natural gas produced is represented by the stream 50.

### Energy Optimization by Means of the Exchanger 15

In the two embodiments (ethane discard and recovery), the use of an exchanger 15, for vaporizing a portion 51 of the liquid of the column 13, integrated with the condensation of a portion 4 of the feed gas 1 and of a partial vaporization of the liquid fraction 19 makes it possible to decrease the energy consumption while at the same time using an exchanger that is relatively simple to design using the brazed aluminum exchanger technology. This exchanger has no two-phase inlet and exhibits temperature differences between hot fluids and cold fluids of less than 30° C. at any place in the exchanger. These two important characteristics make the brazed aluminum technology entirely compatible with the requirements.

This additional thermal integration, coupled with the characteristics of the process which is the subject of the present invention in ethane recovery mode, makes it possible to achieve very high ethane recoveries (for example more than 95%) normally (in the prior art) carried out using recycling of a portion of the compressed gas produced 50.

The following tables summarize the conditions for implementing the embodiments of the process according to the invention of FIGS. 1 and 2.

TABLE 1
Material balance of the process of FIG.
1 in “ethane recovery” mode:

| | Stream 1 | Stream 28 | Stream 50 |
|---|---|---|---|
| C1 | 17 100.00 | 36.30 | 17 063.71 |
| C2 | 1000.00 | 968.28 | 31.74 |
| C3 | 500.00 | 499.37 | 0.63 |
| iC4 | 120.00 | 119.99 | 0.01 |
| nC4 | 200.00 | 199.99 | 0.01 |
| iC5 | 100.00 | 100.00 | 0.00 |
| nC5 | 80.00 | 80.00 | 0.00 |
| nC6 | 40.00 | 40.00 | 0.00 |
| nC7 | 18.00 | 18.00 | 0.00 |
| N₂ | 800.00 | 0.00 | 800.00 |
| H₂S | 0.30 | 0.29 | 0.01 |
| CO₂ | 40.00 | 28.25 | 11.75 |
| COS | 0.70 | 0.70 | 0.00 |
| CH₃—S | 1.00 | 1.00 | 0.00 |
| Total | 20 000.00 | 2092.16 | 17 907.87 |

Composition in kgmol/h

TABLE 2
Operating conditions of the process of FIG.
1 in “ethane recovery” mode:

| | Vapor | Temperature | Pressure | Molar flow rate |
|---|---|---|---|---|
| Stream | fraction | (° C.) | (bara) | (kgmol/h) |
| 1 | 1.000 | 35.0 | 68.0 | 20 000 |
| 5 | 0.896 | −40.1 | 66.5 | 20 000 |
| 7 | 1.000 | −40.1 | 66.5 | 17 913 |
| 8 | 0.000 | −40.1 | 66.5 | 2087 |
| 10 | 1.000 | −40.1 | 66.5 | 12 244 |
| 9 | 1.000 | −40.1 | 66.5 | 5670 |
| 12 | 0.917 | −86.7 | 22.1 | 12 244 |
| 43 | 0.164 | −75.2 | 45.0 | 1580 |
| 8 | 0.387 | −64.1 | 22.1 | 2087 |
| 17 | 1.000 | −105.4 | 21.9 | 17 908 |
| 19 | 0.000 | −0.4 | 22.1 | 2592 |
| 50 | 1.000 | 35.0 | 25.9 | 17 908 |
| 34 | 0.160 | −40.0 | 22.6 | 500 |
| 8″ | 0.345 | −59.7 | 22.1 | 2587 |
| 2 | 1.000 | 35.0 | 68.0 | 11 000 |
| 4 | 1.000 | 35.0 | 68.0 | 9000 |
| 26 | 1.000 | 6.0 | 23.1 | 505 |
| 33 | 1.000 | 4.5 | 23.1 | 500 |
| 28 | 0.000 | 24.8 | 23.3 | 2092 |
| 28 | 0.000 | 4.5 | 23.1 | 5 |
| 23 | 0.000 | −0.3 | 23.3 | 1218 |
| 22 | 0.000 | −0.3 | 23.3 | 1374 |
| 22′ | 0.382 | 29.4 | 23.3 | 1374 |
| 20 | 1.000 | −105.4 | 21.9 | 17 102 |
| 31 | 1.000 | −105.4 | 21.9 | 806 |
| 38 | 0.721 | −76.2 | 46.0 | 5670 |
| 40 | 1.000 | −76.2 | 46.0 | 4090 |
| 41 | 0.000 | −76.2 | 46.0 | 1580 |
| 42 | 0.000 | −103.0 | 45.0 | 4090 |

TABLE 3
Material balance of the process of FIG.
1 in “ethane discard” mode:

| | Stream 1 | Stream 28 | Stream 50 |
|---|---|---|---|
| C1 | 17 100.00 | 0.00 | 17 099.97 |
| C2 | 1000.00 | 14.85 | 985.19 |
| C3 | 500.00 | 495.01 | 4.99 |
| iC4 | 120.00 | 120.00 | 0.00 |
| nC4 | 200.00 | 200.00 | 0.00 |
| iC5 | 100.00 | 100.00 | 0.00 |
| nC5 | 80.00 | 80.00 | 0.00 |
| nC6 | 40.00 | 40.00 | 0.00 |
| nC7 | 18.00 | 18.00 | 0.00 |
| N₂ | 800.00 | 0.00 | 800.00 |
| H₂S | 0.30 | 0.01 | 0.29 |
| CO₂ | 40.00 | 0.00 | 40.00 |
| COS | 0.70 | 0.59 | 0.11 |
| CH₃—S | 1.00 | 1.00 | 0.00 |
| Total | 20 000.00 | 1069.46 | 18 930.56 |

Composition (kgmol/h)

TABLE 4
Operating conditions of the process of FIG.
1 in “ethane discard” mode:

| | Vapor | Temperature | Pressure | Molar flow rate |
|---|---|---|---|---|
| Stream | fraction | (° C.) | (bara) | (kgmol/h) |
| 1 | 1.000 | 35.0 | 68.0 | 20 000 |
| 5 | 0.906 | −37.6 | 66.5 | 20 000 |
| 7 | 1.000 | −37.6 | 66.5 | 18 112 |
| 8 | 0.000 | −37.6 | 66.5 | 1888 |
| 10 | 1.000 | −37.6 | 66.5 | 18 112 |
| 9 | 1.000 | −38.2 | 66.5 | 0 |
| 12 | 0.933 | −75.9 | 28.2 | 18 112 |
| 43 | 0.000 | −78.0 | 54.0 | 0 |
| 8′ | 0.333 | −56.2 | 28.2 | 1888 |
| 17 | 1.000 | −80.4 | 28.0 | 18 931 |
| 19 | 0.000 | −9.2 | 28.2 | 1858 |
| 50 | 1.000 | 35.0 | 34.4 | 18 931 |
| 34 | 0.000 | −75.5 | 28.7 | 788 |
| 8″ | 0.333 | −56.2 | 28.2 | 1888 |
| 2 | 1.000 | 35.0 | 68.0 | 16 000 |
| 4 | 1.000 | 35.0 | 68.0 | 4000 |
| 26 | 1.000 | −4.9 | 29.2 | 1772 |
| 33 | 1.000 | −14.4 | 29.2 | 788 |
| 28 | 0.000 | 110.3 | 29.4 | 1069 |
| 29 | 0.000 | −14.4 | 29.2 | 983 |
| 37 | 0.000 | −75.5 | 28.2 | 788 |
| 23 | 0.000 | −11.1 | 29.5 | 0 |
| 22 | 0.000 | −9.1 | 29.4 | 1858 |
| 22′ | 0.145 | 21.7 | 29.4 | 1858 |
| 20 | 1.000 | −80.4 | 28.0 | 13 983 |
| 31 | 1.000 | −80.4 | 28.0 | 4947 |
| 38 | 0.000 | −77.0 | 55.0 | 0 |
| 40 | 1.000 | −77.0 | 55.0 | 0 |
| 41 | 0.000 | −77.0 | 55.0 | 0 |
| 42 | 0.081 | −75.5 | 54.0 | 0 |
| 37′ | 0.000 | −75.5 | 28.2 | 788 |

TABLE 5
Material balance of the process of FIG.
2 in “ethane recovery” mode:

| | Stream 1 | Stream 28 | Stream 50 |
|---|---|---|---|
| C1 | 17 100.00 | 36.55 | 17 063.45 |
| C2 | 1000.00 | 975.01 | 24.98 |
| C3 | 500.00 | 499.66 | 0.33 |
| iC4 | 120.00 | 119.99 | 0.00 |
| nC4 | 200.00 | 200.00 | 0.00 |
| iC5 | 100.00 | 100.00 | 0.00 |
| nC5 | 80.00 | 80.00 | 0.00 |
| nC6 | 40.00 | 40.00 | 0.00 |
| nC7 | 18.00 | 18.00 | 0.00 |
| N₂ | 800.00 | 0.00 | 800.00 |
| H₂S | 0.30 | 0.29 | 0.01 |
| CO₂ | 40.00 | 28.03 | 11.97 |
| COS | 0.70 | 0.70 | 0.00 |
| CH₃—S | 1.00 | 1.00 | 0.00 |
| Total | 20 000.00 | 2099.24 | 17 900.75 |

Composition (kgmol/h)

TABLE 6
Operating conditions of the process of FIG.
2 in “ethane recovery” mode:

| | Vapor | Temperature | Pressure | Molar flow rate |
|---|---|---|---|---|
| Stream | fraction | (° C.) | (bara) | (kgmol/h) |
| 1 | 1.000 | 35.0 | 68.0 | 20 000 |
| 5 | 0.892 | −41.0 | 66.5 | 20 000 |
| 7 | 1.000 | −41.0 | 66.5 | 17 835 |
| 8 | 0.000 | −41.0 | 66.5 | 2165 |
| 10 | 1.000 | −41.0 | 66.5 | 12 306 |
| 9 | 1.000 | −41.0 | 66.5 | 5529 |
| 12 | 0.914 | −87.4 | 22.2 | 12 306 |
| 43 | 0.000 | −71.7 | 61.2 | 521 |
| 8′ | 0.390 | −65.3 | 22.2 | 2165 |
| 17 | 1.000 | −105.7 | 22.0 | 17 901 |
| 19 | 0.000 | −2.2 | 22.2 | 2595 |
| 50 | 1.000 | 35.0 | 67.5 | 17 901 |
| 34 | 0.000 | −59.0 | 22.7 | 496 |
| 8″ | 0.323 | −65.2 | 22.2 | 2655 |
| 2 | 1.000 | 35.0 | 68.0 | 11 000 |
| 4 | 1.000 | 35.0 | 68.0 | 9000 |
| 26 | 1.000 | 3.9 | 23.2 | 501 |
| 33 | 1.000 | 2.3 | 23.2 | 496 |
| 28 | 0.000 | 24.7 | 23.4 | 2099 |
| 29 | 0.000 | 2.3 | 23.2 | 5 |
| 23 | 0.000 | −2.1 | 23.4 | 1349 |
| 22 | 0.000 | −2.1 | 23.4 | 1246 |
| 22′ | 0.442 | 32.5 | 23.4 | 1246 |
| 20 | 1.000 | −105.7 | 22.0 | 17 095 |
| 31 | 1.000 | −105.7 | 22.0 | 806 |
| 38 | 0.906 | −62.1 | 62.2 | 5529 |
| 40 | 1.000 | −62.1 | 62.2 | 5008 |
| 41 | 0.000 | −62.1 | 62.2 | 521 |
| 44 | 0.604 | −83.6 | 41.0 | 5008 |
| 46 | 1.000 | −83.6 | 41.0 | 3025 |
| 47 | 0.000 | −83.6 | 41.0 | 1982 |
| 49 | 0.000 | −100.0 | 40.5 | 1982 |

The process which is the subject of the present invention uses a turboexpander 11 and two fractionation columns 13 and 24 linked to heat exchangers 3, 15, 30. The exchangers which provide the precooling of the natural gas before it is expanded in the turboexpander and also the condensation of the gas streams serving as reflux can consist of one or more brazed aluminum plate exchanger bodies designed in a specific way to avoid any two-phase distribution of refrigerant.

The process which is the subject of the present invention makes it possible to obtain, in the preferred embodiment thereof, a degree of propane recovery of greater than 99.5% and an adjustable degree of ethane recovery of 0 to more than 95%. It does not require any recycling of treated gas, which makes it particularly advantageous when the gas is intended for a denitrogenation unit. Given that the degree of propane recovery is very high, this process also makes it possible to remove, from the natural gas, carbonyl sulfide (COS) and also the other sulfur-bearing impurities, such as methyl mercaptan (CH₃SH).

It can be carried out in several different ways according to the choice of arrangement of the feeds of the first column 13 and of the arrangement of the condenser of the second column 24. The process does not use lateral reboilers, which facilitates the installation thereof and the operation thereof.

For the brazed aluminum exchangers, it is often necessary to separate the liquid and vapor phases using a drum for mixing them at the inlet of each passage of each body. This is complicated and expensive. The distribution thus performed is not perfect. It is therefore necessary to overcome this major process drawback in another way. One known technique consists in separating the phases and in injecting them separately into the heat exchangers. The processes must be adjusted, but this results in a loss of thermodynamic efficiency. The new process not using two-phase distributions is not confronted with these problems.

It will be understood that many additional changes in the details, materials, steps and arrangement of parts, which have been herein described in order to explain the nature of the invention, may be made by those skilled in the art within the principle and scope of the invention as expressed in the appended claims. Thus, the present invention is not intended to be limited to the specific embodiments in the examples given above.

What is claimed is:

1. A process for simultaneously producing treated natural gas and a propane-rich stream from a feed gas comprising methane, ethane and hydrocarbons having more than three carbon atoms, said process comprising the following steps:
Step a): cooling and partially condensing the feed gas;
Step b): separating the cooled gas resulting from step a) into a first liquid stream and a first gas stream by means of a first phase separator vessel at a temperature T1 and a pressure P1;
Step c): expanding at least one portion of the first gas stream resulting from step b) using an expansion means;
Step d): introducing the expanded gas resulting from step c) into a first distillation column at a first intermediate level N′;
Step e): recovering a bottom liquid fraction from said first distillation column and introducing the recovered bottom liquid fraction into a second distillation column at a feed level M1;
further comprising exclusively one or the other of the following steps depending on the desired degree of ethane in the streams produced:
Step f): partially condensing and introducing into a second phase separator vessel at a pressure P2 and a temperature T2 in order to produce a second gas stream and a second liquid stream, and condensing and introducing at least one portion of said second gas stream into said first distillation column at a level S1 above the level N′, in order to obtain degrees of ethane extraction greater than a first predetermined threshold;
Step g): recovering a gas fraction from the top of said second distillation column and condensing this gas fraction before being introduced into said first distillation column at the level S1 above the level N′, in order to obtain degrees of ethane extraction below a second predetermined threshold.

2. The process according to the claim 1, wherein P2 is lower than P1 and T2 is lower than T1.

3. The process according to claim 1, wherein step f) also comprises step f1): partially condensing at least one portion of said second gas stream resulting from the second phase separator vessel and introducing the partially condensed one portion of said second gas into a third phase separator vessel at a pressure P3 and a temperature T3 in order to produce a third gas stream and a third liquid stream; condensing at least one portion of said third gas stream and introduced into said first distillation column at the level S1 above the level N′.

4. The process according to claim 3, wherein P1<P2<P3 and T1<T2<T3.

5. The process according to claim 1, wherein said first predetermined threshold is greater than or equal to 80%.

6. The process according to claim 1, wherein said second predetermined threshold is less than or equal to 20%.

7. The process according to claim 1, wherein said propane-rich stream comprises at least 99.5% of the propane initially contained in the feed stream.

8. The process according to claim 1, wherein said ethane-rich stream comprises at least 95% of the ethane initially contained in the feed stream.

9. The process according to claim 1, wherein a portion of the gas fraction from the top of the second distillation column is condensed in a heat exchanger by circulation of a portion of the gas from the top of the first distillation column.

10. The process according to claim 1, wherein:
during step a), the feed gas is at least partially condensed in a first heat exchanger; a liquid stream is extracted from the first distillation column at an intermediate level S2 lower than the level N″ and is partially vaporized in a second heat exchanger distinct from said first heat exchanger;
said liquid fraction recovered during step e) is pumped into and then at least partially vaporized in said second heat exchanger; and
a fraction of the feed gas is cooled in said second heat exchanger.

11. A facility, for carrying out the process defined in claim 1, for simultaneously producing treated natural gas and a propane-rich stream from a feed gas comprising methane, ethane and hydrocarbons having more than three carbon atoms, said facility comprising:
a first heat exchanger for cooling to condense a feed gas;
a first phase separator vessel for separating the gas cooled in the first condensation means into a first liquid stream and a first gas stream;
a first distillation column into which at least one portion of the first gas stream is introduced at a first intermediate level N′;
a second distillation column into which a liquid fraction originating from the bottom of said first distillation column is introduced at least one feed level M1, M2;
further comprising a means for producing a stream, having a degree of ethane recovery above a predetermined threshold, originating from a second phase separator vessel, located downstream of the first phase separator vessel, producing a second gas stream and a second liquid stream, at least one portion of said second gas stream being condensed and introduced into said first distillation column at a level S1 above the level N′; and
further comprising a means for producing a stream, having a degree of ethane recovery below a second predetermined threshold, originating from a gas fraction at the top of said second distillation column, then introduced into said first distillation column at the level S1 above the level N′.

12. The facility according to claim 11, further comprising a third phase separator vessel, located downstream of the second phase separator vessel, producing a third gas stream and a third liquid stream, at least one portion of said third gas stream being condensed and introduced into said first distillation column at the level S1 above the level N′.

13. The facility according to claim 11, further comprising a second heat exchanger capable of and designed for:
partially vaporizing a liquid stream extracted from the first distillation column at an intermediate level S2 lower than the level N″ and also a liquid fraction recovered at the bottom of said first distillation column; and
cooling and at least partially condensing a fraction of the feed gas.
